(19) United States
(12) Patent Application Publication
(10) Pub. No.: US 2003/0105047 A1
(43) Pub. Date: Jun. 5, 2003

(54) Techniques for treating neurodegenerative disorders by brain infusion of mutational vectors

(75) Inventors: William F. Kaemmerer, Edina, MN (US); Christopher D. Keene, Minneapolis, MN (US); Betsy J. Kren, Minneapolis, MN (US); Walter C. Low, Excelsior, MN (US); Clifford J. Steer, St. Paul, MN (US)

Correspondence Address: BANNER & WITCOFF, LTD., TEN SOUTH WACKER DRIVE, SUITE 3000, CHICAGO, IL, 60606, US

(73) Assignee: Medtronic, Inc., Minneapolis, MN

(21) Appl. No.: 10/133,957

(22) Filed: Apr. 26, 2002

Related U.S. Application Data
(60) Provisional application No. 60/334,377, filed on Nov. 30, 2001.

(51) Int. Cl. A61K 48/00; C12N 15/85; A61M 31/00

(52) U.S. Cl. 514/44; 604/500; 604/890.1; 435/455

(57) ABSTRACT

A method is disclosed for treating a neurodegenerative disorder comprising the steps of surgically implanting a catheter so that a discharge portion of the catheter lies adjacent a predetermined infusion site in a brain, and discharging through the discharge portion of the catheter a predetermined dosage of at least one substance to the infusion site of the brain, the at least one substance capable of altering a nucleotide in a DNA sequence of a gene to convert a codon in a protein-coding region of the gene into a stop codon in the brain, whereby neurodegeneration in the brain is reduced. In a preferred embodiment, the at least one substance is a mutational vector, for example, a RNA/DNA chimeric mutational vector. The disclosed invention provides a method for treating neurodegenerative disorders such as Huntington's disease, spinocerebellar ataxia type 1, type 2, type 3, type 6, and/or type 7, spinobulbar muscular atrophy (Kennedy's disease), and/or dentatorubral-pallidoluysian atrophy (DRPLA).

### FIELD OF INVENTION

This invention relates to techniques for treating neurodegenerative disorders by brain infusion of mutational vectors.

### BACKGROUND OF THE INVENTION

Several neurodegenerative diseases, including Huntington's disease and various types of hereditary ataxia, are each known to be caused by genetic mutations that result in the production of a corresponding mutant protein with a new, pathogenic function. There is currently no technique to alter the DNA within cells in vivo that results in a cure for Huntington's disease or the other hereditary neurodegenerative diseases. These diseases are progressively debilitating and ultimately fatal.

The design and use of chimeric mutational vectors to effect mutation in a target gene of a eukaryotic cell by homologous recombination is disclosed in U.S. Pat. Nos. 5,731,181 and 5,795,972. U.S. Pat. No. 5,731,181 states that other applications of the invention include the introduction of stop codons.

U.S. Pat. Nos. 6,004,804 and 6,010,907 disclose a method and use of non-chimeric mutational vectors. Non-chimeric mutational vectors do not have an RNA:DNA hybrid-duplex region that is a characteristic of chimeric mutational vectors.

None of the above four patents disclose methods for the successful delivery of mutational vectors to targeted cells in a manner capable of accomplishing treatment of neurogenerative diseases by changing a nucleotide in the DNA sequence of a gene. The above patents do not disclose use of delivery devices or any method of delivery or infusion of mutational vectors to the central nervous system (“CNS”). For example, the above patents do not disclose or suggest a method of delivery or infusion of mutational vectors to the CNS by an implantable device, catheter, or stereotactic surgery.

Further, these patents do not disclose any technique for infusing into the brain mutational vectors, nor do they disclose whether mutational vectors, upon infusion into the brain, are capable of entering neurons and traveling to the nucleus of targeted cells, whereupon a codon in a protein-coding region of a mutant gene can be converted into a stop codon, and thus prevent production of a pathogenic protein by the mutant gene.

Systemic delivery of oligonucleotides is neither possible nor desirable. Oligonucleotides will not persist in vivo long enough to enable oral or intravenous administration, nor are they likely to cross the blood-brain barrier.

An alternative delivery of oligonucleotides by brain infusion may be the injection of oligonucleotides into the cerebral arteries accompanied by pharmaceutical agents known to temporarily disrupt the blood-brain barrier. However, this approach may be impractical because of the large quantity of oligonucleotide that might have to be administered by this method to achieve an effective quantity in the brain. Even when the blood-brain barrier is temporarily opened, the vast majority of oligonucleotide delivered via the bloodstream may be lost to other organ systems in the body, especially the liver.

Furthermore, some of the proteins involved in neurodegenerative diseases perform essential functions elsewhere in the body, despite the presence of the mutation. For example, the Huntington's protein has been found to be essential for the production of blood cells (see Metzler, M., Helgason, C., Dragatsis, I., Zhang, T., Gan, L., Pineult, N., Zeitlin, S., Humpheries, R., and Hayden, M., “Huntington is required for normal hematopoiesis,” Hum. Mol. Genet. 9: 387-94 (2000)). Therefore, it would not be appropriate to prevent production of the protein in other cells beyond those at risk for neurodegeneration. Thus, administration of large amounts of oligonucleotide into the bloodstream, as likely would be necessary using the blood-brain barrier disruption approach, may have unacceptable risks and side effects.

U.S. Pat. Nos. 5,735,814 and 6,042,579 disclose the use of drug infusion for the treatment of Huntington's disease, but the drugs specifically identified in these patents pertain to agents capable of altering the level of excitation of neurons, and do not specifically identify agents intended to alter the DNA within cells.

### SUMMARY OF THE INVENTION

The present invention comprises a method of treating a neurodegenerative disorder comprising the steps of surgically implanting an intraparenchymal catheter having a port so that a discharge portion of the catheter lies adjacent a predetermined infusion site in a brain, and discharging through the discharge portion of the catheter a predetermined dosage of at least one substance to the infusion site of the brain, the at least one substance capable of altering a nucleotide in a DNA sequence of a gene to convert a codon in a protein-coding region of the gene into a stop codon in the brain, whereby neurodegeneration in the brain is reduced.

In a preferred embodiment, the at least one substance is a mutational vector, for example, a RNA/DNA chimeric mutational vector. The disclosed invention provides a method for treating neurodegenerative disorders such as Huntington's disease, spinocerebellar ataxia type 1, type 2, type 3, type 6, and/or type 7, spinobulbar muscular atrophy (Kennedy's disease), and/or dentatorubral-pallidoluysian atrophy (DRPLA).

Thus, the present invention provides techniques to treat neurodegenerative diseases by preventing production of a pathogenic protein by introducing a change in the corresponding gene. In particular, the present invention provides methods of infusing mutational vectors (i.e., synthetic oligonucleotides) to a target area of a patient to change a gene by inserting, deleting or altering a nucleotide in the DNA sequence of the gene to convert a codon in the protein-coding region of the gene into a stop codon, and thus prevent production of a pathogenic protein by the gene.

### BRIEF DESCRIPTION OF THE DRAWINGS

5 FIG. 1 shows a sagittal section of the cerebellum of a mouse injected 20 hours earlier with 2 microliters of mutational vectors (more specifically in this example, chimeraplasts) at 6 micrograms per microliter. FIG. 1 shows the section under phase contrast illumination using a 10× microscope objective. The significance of this photograph is the evidence of where the injection needle's tip was located when the injection was made. The dark spots in the center of the photograph are granules of charcoal left behind by the tip of the infusion needle. The white circles in the left of the photograph are the cell bodies of Purkinje neurons.

FIG. 2 shows the same section of mouse brain tissue as shown in FIG. 1, but under fluorescent illumination. The fluorescence reveals the fluorescent molecular tag on the chimeraplast preparation. The significance of this photograph is that it shows uptake of the chimeraplast molecules by Purkinje neurons, evident from the fluorescent signal coming from the location of the Purkinje neurons (compare to FIG. 1).

FIG. 3 is another sagittal section of cerebellar tissue from the same mouse as FIGS. 1 and 2, under fluorescent illumination using lower magnification (a 4× microscope objective). The dark spots in the middle right of the photograph are charcoal remnants that show the angle of entry of the injection needle through the brain tissue. Fluorescence shows that much of the injected chimeraplast solution diffused out of the brain up a sulcus (brain convolution). The significance of this photograph is that it shows that nevertheless, substantial uptake of chimeraplasts into Purkinje neurons occurred. This can be seen by observing the row of fluorescent spots, consisting of signals coming from Purkinje cell bodies that surround the more intense signal coming from the sulcus.

FIG. 4 is a higher magnification view of a portion of the same tissue section as photographed in FIG. 3, this time using a 40× microscope objective and fluorescent illumination. This figure shows a concentration of the fluorescent signal within central regions of Purkinje neurons. The significance of this figure is that it shows evidence suggesting that the chimeraplast molecules entered the nuclei of the Purkinje cells.

FIG. 5 is a medium magnification view (20× microscope objective) of yet another tissue section from the same mouse cerebellum, under fluorescent illumination. This significance of this figure is that it shows entry of chimeraplasts into numerous Purkinje neurons and apparent transport of chimeraplasts into these cell's nuclei.

FIG. 6 is an additional view of Purkinje neurons in the same tissue section as photographed in FIG. 5, this time using a 40× microscope objective and fluorescent illumination. This figure provides additional evidence suggesting that the chimeraplasts have entered the nuclei of the Purkinje cells.

FIGS. 7, 8, and 9 are three views of the same sagittal section of mouse cerebellum, from the same mouse as portrayed in FIGS. 1 through 6. FIG. 7 shows the tissue section under fluorescent illumination, using a 20× microscope objective. The significance of FIG. 7 is that it shows the position of the signal from the fluorescein-labeled chimeraplasts.

FIG. 8 is the same tissue section as FIG. 7, under fluorescent illumination for the Cy-3 fluorophore. This tissue section has been immunostained for calbindin, a marker for Purkinje neurons, using a primary antibody against calbindin and a Cy-3 conjugated secondary antibody. The significance of this photograph is that it identifies the Purkinje neurons in the tissue by virtue of the Cy-3 signal.

FIG. 9 is the superimposition of FIGS. 7 and 8, indicating that the position of the signals from the fluorescein-labelled chimeraplasts and the signals from the Cy-3/calbindin antibodies are located in the same place. The significance of this figure is that it provides evidence that the neurons that were entered by the chimeraplasts are Purkinje neurons.

FIG. 10 is a sagittal section of cerebellar tissue from a mouse that had been injected 20 hours earlier with 2 microliters of chimeraplasts at a concentration of 0.6 micrograms per microliter. The significance of this figure is the position of the injection, which can be seen to have been in the molecular (outer) layer of the cerebellar tissue, and the absence of a punctate signal obtained from neurons, indicating that few neurons took up the chimeraplasts when the injection site was in the outer layer of the tissue.

FIG. 11 is a sagittal section of cerebellar tissue from a mouse that had been injected 20 hours earlier with 2 microliters of chimeraplasts at a concentration of 0.06 micrograms per microliter (which is 10 times less than the mouse portrayed in FIG. 10). This photograph, taken using fluorescent illumination and a 10× microscope objective, shows that even at this low concentration, uptake of chimeraplasts into Purkinje neurons is evident. Together with FIG. 10, this figure suggests that the site of injection of the chimeraplasts within the brain tissue can alter the likelihood that the chimeraplasts will enter specific neuronal cell populations.

FIG. 12 is a sagittal section of cerebellar tissue from a mouse that had been injected 20 hours earlier with 2 microliters of chimeraplasts at a concentration of 0.06 micrograms per microliter (same as portrayed in FIG. 11). This figure indicates that even at this low concentration, chimeraplasts were taken up by substantial numbers of Purkinje neurons.

FIG. 13 shows a section of brain tissue from a mouse that had been injected 20 hours earlier with 2 microliters of chimeraplasts at a concentration of 6.0 micrograms per microliter into the striatum. This photograph, taken using fluorescent illumination, indicates that chimeraplasts can be taken up by neurons in the striatum when the striatum is the site of the injection of the chimeraplasts.

FIG. 14 shows a section of brain tissue from the same mouse as portrayed in FIG. 13, using fluorescent illumination, but using a higher power microscope objective. This figure indicates that the chimeraplasts injected into the striatum are taken up by neurons.

FIG. 15 is a schematic illustration of an example of a catheter for use in a preferred embodiment of the present invention.

FIG. 16 is a schematic illustration of the catheter shown in FIG. 15 when surgically implanted in a patient.

### DESCRIPTION OF THE PREFERRED EMBODIMENTS

The present invention solves two problems in the prior art at the same time: (1) the problem of how to treat neurodegenerative diseases caused by the production in neurons of a protein that has pathogenic properties, for example due to a genetic mutation; and (2) the problem of delivery of therapeutic oligonucleotides to affected neurons.

In accordance with the present invention, oligonucleotides are designed as mutational vectors against specific genes to prevent the production of the disease-causing proteins in neurons. Animal tests in accordance with the present invention demonstrated that the designed oligonucleotides can be successfully delivered to targeted cells within the brain of an animal. The successful animal tests are indicative that the designed oligonucleotides can be successfully delivered to the human central nervous system and human brain to treat neurogenerative diseases.

Mutational vectors are synthetic oligonucleotides that have been shown to be capable of introducing nucleotide changes into cells both in vitro (see Kren, B., Cole-Strauss, A., Kmiec, E., and Steer, C., “Targeted nucleotide exchange in the alkaline phophatase gene of HuH-7 cells mediated by a chimeric RNA/DNA oligonucleotide,” Hepatology 25: 1462-8 (1997)), and in vivo (see Kren, B., Bandyopadhyay, P., and Steer, C., “In vivo site-directed mutagensis of the factor IX gene by chimeric RNA/DNA oligonucleotides,” Nature Medicine 4: 285-90 (1998)). Additional work has shown that RNA/DNA chimeric mutational vectors (also known as “chimeraplasts”) can correct an inherited single-base mutation in the gene for an essential liver enzyme in an animal model of Crigler-Najjar syndrome (see Kren, B., Parashar, B., Bandyopadhyay, P., Chowdhury, N., Chowdhury, J., and Steer, C., “Correction of the UDP-glucuronosyltransferase gene defect in the Gunn rat model of Crigler-Najjar syndrome type I with a chimeric oligonucleotide,” Proc. Natl. Acad. Sci. USA 96: 10349-54 (1999)).

The present invention provides a delivery system for a mutational vector therapy for neurodegenerative diseases that permits delivery of repeated bolus injections of high concentrations of oligonucleotides to multiple sites in the CNS over an extended period of care for the patient, for example years.

In a preferred embodiment, RNA/DNA chimeric mutational vectors (also known as “chimeraplasts”) are surgically injected into the brain, are taken up by neurons and transported to the nucleus of targeted cells, and trigger a change in the targeted cell DNA that prevents production of a pathogenic protein.

The present invention provides methods of using neurosurgical devices to deliver therapeutic mutational vectors to the central nervous system of patients. In particular, the present invention provides methods that use surgically implanted catheters to repeatedly or chronically deliver mutational vectors to the brain. The mutational chimeraplasts introduce a new stop codon into the targeted gene, thereby stopping the production of a disease-causing protein.

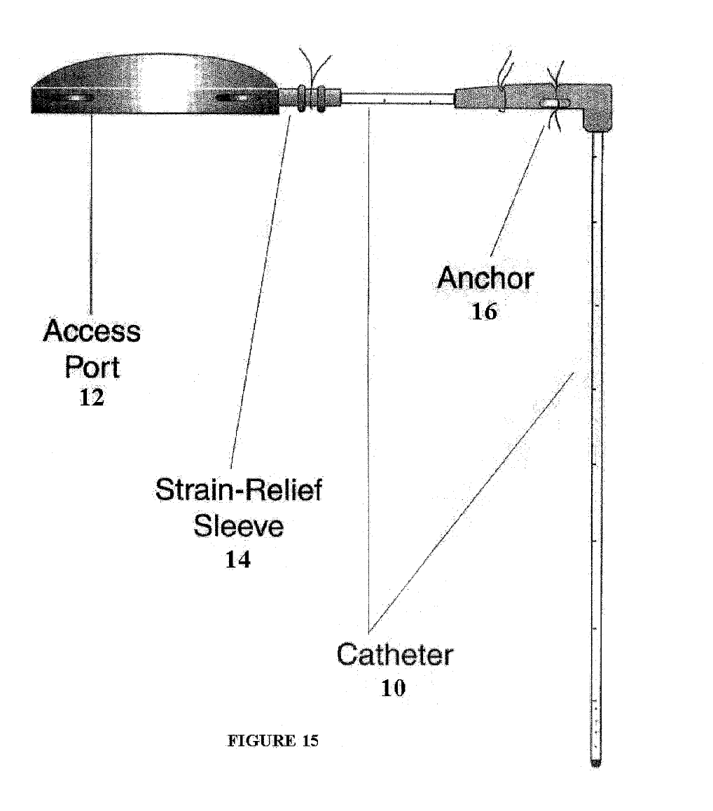

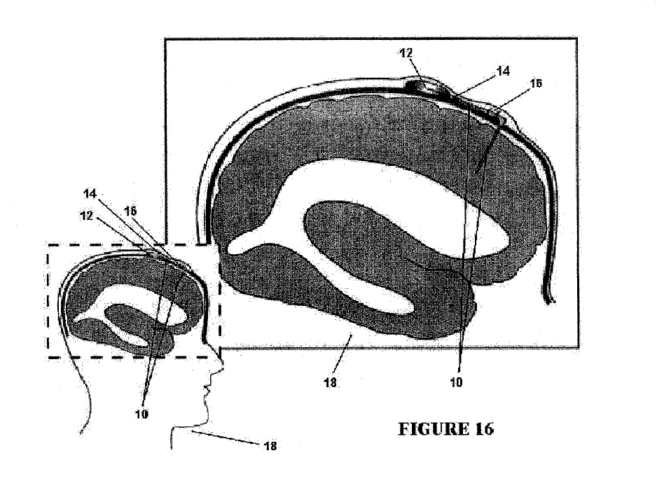

Devices and systems can be used in accordance with the present invention to infuse mutational vectors, including devices and systems intended for infusion of substances into the central nervous system. Examples include the Model 8506 investigational device (by Medtronic, Inc. of Minneapolis, Minn.), which can be implanted subcutaneously on the cranium, and provides an access port through which therapeutic agents may be delivered to the brain. Delivery occurs through a stereotactically implanted polyurethane catheter. The Model 8506 is schematically depicted in FIGS. 15 and 16. Two models of catheter that can function with the Model 8506 access port include the Model 8770 ventricular catheter by Medtronic, Inc., for delivery to the intracerebral ventricles, which is disclosed in U.S. Pat. No. 6,093,180, incorporated herein by reference, and the IPA1 catheter by Medtronic, Inc., for delivery to the brain tissue itself (i.e., intraparenchymal delivery), disclosed in U.S. Ser. Nos. 09/540,444 and 09/625,751, which are incorporated herein by reference. The latter catheter has multiple outlets on its distal end to deliver the therapeutic agent to multiple sites along the catheter path. Those of skill in the art will recognize that these and other devices and systems may be suitable for delivery of mutational vectors for the treatment of neurodegenerative diseases in accordance with the present invention.

In one preferred embodiment, the method further comprises the steps of implanting a pump outside the brain, the pump coupled to a proximal end of the catheter, and operating the pump to deliver the predetermined dosage of the at least one substance through the discharge portion of the catheter. A further embodiment comprises the further step of periodically refreshing a supply of the at least one substance to the pump outside said brain.

The delivery of the mutational vectors in accordance with the present invention can be accomplished with a wide variety of devices, including but not limited to U.S. Pat. Nos. 5,735,814, 5,814,014, and 6,042,579, all of which are incorporated herein by reference.

Thus, the present invention includes the delivery of mutational vectors using an implantable pump and catheter, like that taught in U.S. Pat. No. 5,735,814 and 6,042,579, and further using a sensor as part of the infusion system to regulate the amount of mutational vectors delivered to the brain, like that taught in U.S. Pat. No. 5,814,014.

Other devices and systems can be used in accordance with the method of the present invention, for example, the devices and systems disclosed in U.S. Ser. Nos. 09/872,698 (filed Jun. 1, 2001) and 09/864,646 (filed May 23, 2001), which are incorporated herein by reference.

A mutational vector will prevent production of the pathogenic protein by altering the genetic code for the protein itself. Repeated administration of the therapeutic agent to the patient will likely be required to accomplish the change in a large enough number of neurons to improve the patient's quality of life. Within an individual neuron, however, the change is permanent and further application of the therapeutic agent, while harmless, would not be necessary. In contrast, the alternative approaches to suppressing pathogenic protein production, such as the use of antisense oligonucleotides or ribozymes, require either continuous administration of the therapeutic molecules themselves, or stable transfection of neurons with DNA encoding for the antisense oligonucleotide or ribozyme sequence, and continued expression of that foreign DNA. While it may be possible to accomplish the latter with viral vectors or other biotechnologies, development of successful therapies involving in vivo transfection of neurons may be more challenging than an approach based on delivery of mutational vectors to targeted cells.

The present invention takes into account the following considerations. The native DNA repair system in cells is as likely to alter the oligonucleotide as the genomic DNA, and thus there is a chance that the first time a molecule of the oligonucleotide preparation mates with the target DNA, the oligonucleotide will be altered, not the gene. Such an altered oligonucleotide may then separate from the target gene without having had the intended effect. The next oligonucleotide to come along may or may not have the same fate. However, if a huge number of oligonucleotides enter the cell, eventually the genomic DNA will be altered as intended. In addition, while an oligonucleotide that was itself altered can come back and to undo the desired change in the genomic DNA, oligonucleotides are known to be degraded in cells within 24 to 48 hours. Further, to maximize the number of oligonucleotides getting into cells, so that the desired kinetics as discussed above are favored, the oligonucleotides should be delivered locally to the target cells in a concentrated solution. Also, since oligonucleotide preparations will not cross the blood-brain barrier, the delivery should be local to the CNS. Further, to maximize the number of cells in which the repair occurs, the local delivery should occur at multiple sites.

An alternative strategy may be to deliver the vector to the cerebrospinal fluid (“CSF”), and relying upon the circulation of the CSF to expose the targeted cells to the vector. In this alternative strategy, vector molecules would be targeted to specific cells by conjugating them with ligands for receptors known to exist on the targeted cell population (see Bandyopadhyay, P., Ma, X., Linehan-Stieers, C., Kren, B., and Steer, C., “Nucleotide exchange in genomic DNA of rat hepatocytes using RNA/DNA oligonucleotides- Targeted delivery of liposomes and polyethylenimine to the asialoglycoprotein receptor,” J. Biol. Chem. 274: 10163-72 (1999)). Because the diseases that can be treated with the method of the present invention are chronic and ultimately fatal, repeated injections of the therapeutic formulation should be delivered until the patient's condition improves, or there is other evidence to indicate that sufficient therapy has been delivered.

For the mutational vector therapy for neurodegenerative disease of the present invention, multiple catheters having access ports can be implanted in a given patient for a complete therapy. In a preferred embodiment, there is one port and catheter system per cerebral or cerebellar hemisphere, and perhaps several. Once the implantations are performed by a neurosurgeon, the patient's neurologist can perform a course of therapy consisting of repeated bolus injections of oligonucleotides over a period of weeks to months, along with monitoring for therapeutic effect over time. The devices can remain implanted for several months or years for a full course of therapy. After confirmation of therapeutic efficacy, the access ports might optionally be explanted, and the catheters can be sealed and abandoned, or explanted as well. The device material should not interfere with magnetic resonance imaging, and, of course, the oligonucleotide preparations must be compatible with the access port and catheter materials and any surface coatings.

To summarize, the present invention provides methods to deliver mutational vectors to the human central nervous system, and thus treat neurodegenerative diseases by altering the DNA within neurons to prevent the production of a pathogenic protein.

The present invention is directed for use as a treatment for neurodegenerative disorders and/or diseases, comprising Huntington's disease, spinocerebellar ataxia type 1, type 2, type 3, type 6, and/or type 7, spinobulbar muscular atrophy (Kennedy's disease), and/or dentatorubral-pallidoluysian atrophy (DRPLA), and/or any other neurogenerative disease caused by the gain of a new, pathogenic function by a mutant protein.

### EXAMPLE 1

In accordance with the present invention, RNA/DNA chimeric mutational vectors (also known as “chimeraplasts”) were surgically infused into the brain of a mouse, whereupon it was discovered that the chimeraplasts were taken up by neurons and transported to the nucleus of targeted cells so that they could trigger a change in the targeted cell DNA.

FIGS. 1 through 6 are photographs of mutational vectors (more specifically in this example, chimeraplasts) in neurons within a mouse cerebellum, 20 hours after in vivo infusion of the chimeraplasts into the mouse brain. More specifically, FIGS. 1 through 6 show sagittal W sections of the cerebellar brain tissue, under normal and fluorescent illumination. In the cerebellum, large neurons known as Purkinje cells are arrayed in a row beneath and parallel to the folds (convolutions) of the brain. In spinocerebellar ataxia, dysfunction and degeneration of Purkinje cells are a major cause of the patient's symptoms.

FIG. 1 shows a sagittal section of the cerebellum of a mouse injected 20 hours earlier with 2 microliters of chimeraplasts at 6 micrograms per microliter. FIG. 1 shows the section under phase contrast illumination using a 10× microscope objective. The significance of this photograph is the evidence of where the injection needle's tip was located when the injection was made. The dark spots in the center of the photograph are granules of charcoal left behind by the tip of the infusion needle, used for later identification of the needle's position. The white circles in the left of the photograph are the cell bodies of Purkinje neurons. Purkinje neurons are among the neurons that generate spinocerebellar ataxia type 1, and similar neurodegenerative diseases.

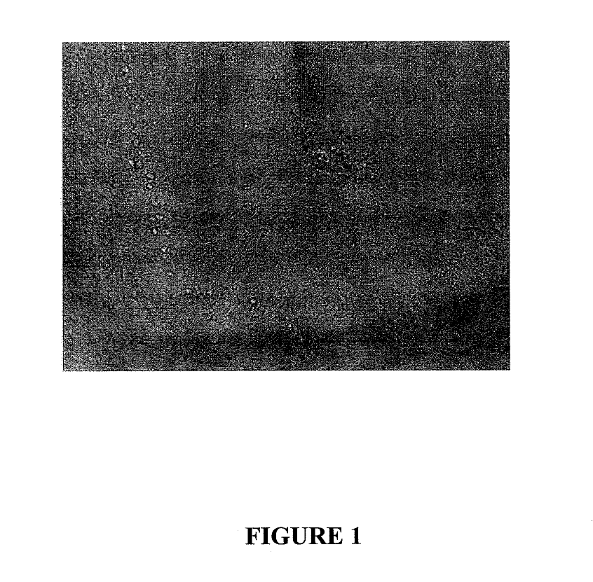

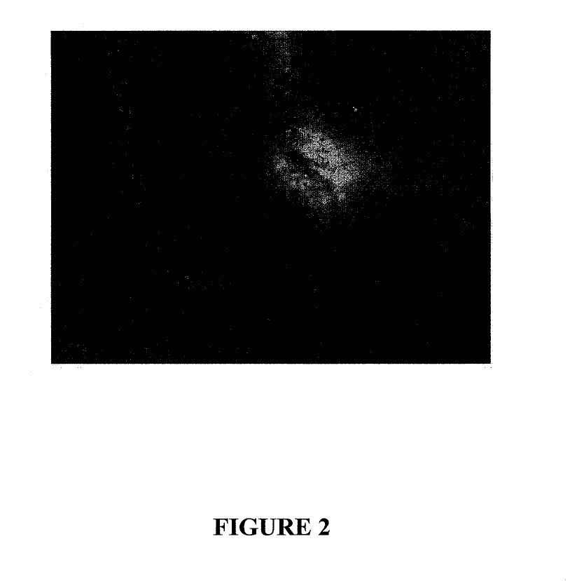

FIG. 2 shows the same section of mouse brain tissue as shown in FIG. 1, but under fluorescent illumination. The fluorescence reveals the fluorescent molecular tag on the chimeraplast preparation. The significance of this photograph is that it shows uptake of the chimeraplast molecules by Purkinje neurons, evident from the fluorescent signal coming from the location of the Purkinje neurons (compare to FIG. 1).

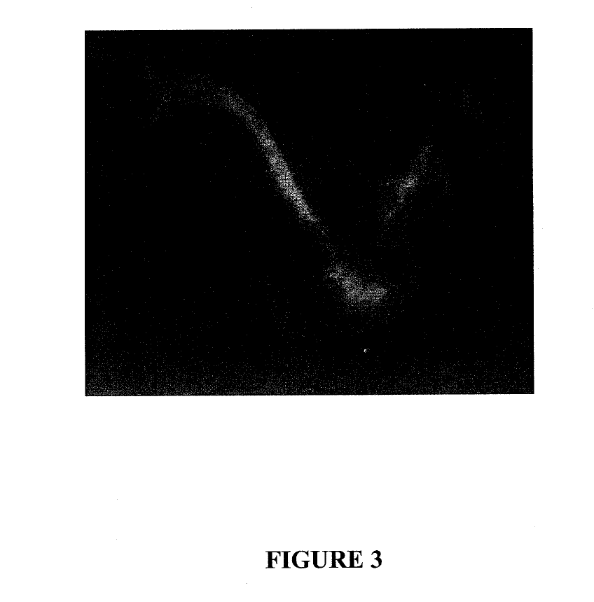

FIG. 3 is another sagittal section of cerebellar tissue from the same mouse as FIGS. 1 and 2, under fluorescent illumination using lower magnification (a 4× microscope objective). The dark spots in the middle right of the photograph are charcoal remnants that show the angle of entry of the injection needle through the brain tissue. Fluorescence shows that much of the injected chimeraplast solution diffused out of the brain up a sulcus (brain convolution). The significance of this photograph is that it shows that nevertheless, substantial uptake of chimeraplasts into Purkinje neurons occurred. This can be seen by observing the row of fluorescent spots, consisting of signals coming from Purkinje cell bodies that surround the more intense signal coming from the sulcus.

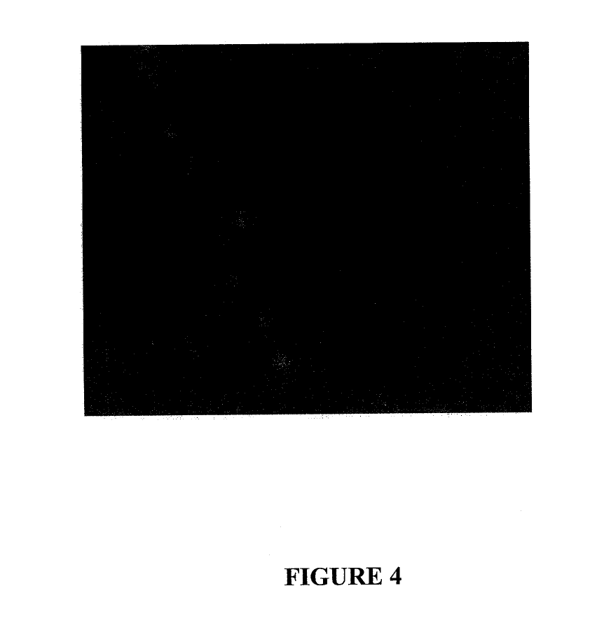

FIG. 4 is a higher magnification view of a portion of the same tissue section as photographed in FIG. 3, this time using a 40× microscope objective and fluorescent illumination. This figure shows a concentration of the fluorescent signal within central regions of Purkinje neurons. The significance of this figure is that it shows evidence suggesting that the chimeraplast molecules entered the nuclei of the Purkinje cells.

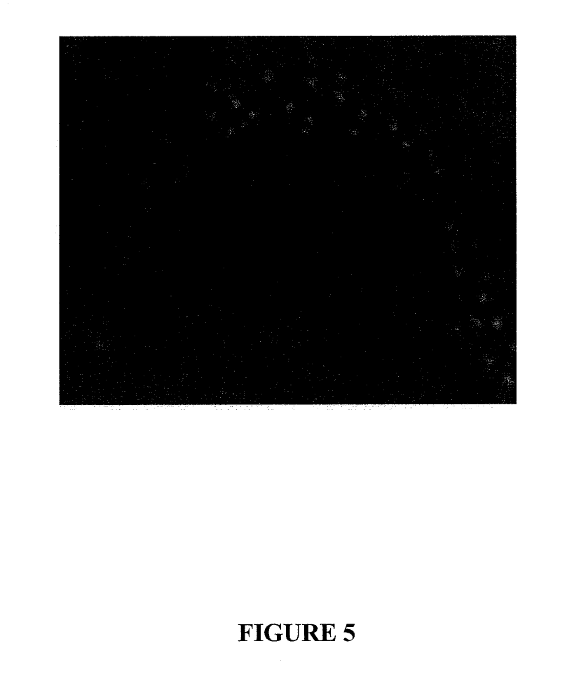

FIG. 5 is a medium magnification view (20× microscope objective) of yet another tissue section from the same mouse cerebellum, under fluorescent illumination. This significance of this figure is that it shows entry of chimeraplasts into numerous Purkinje neurons and apparent transport of chimeraplasts into these cell's nuclei.

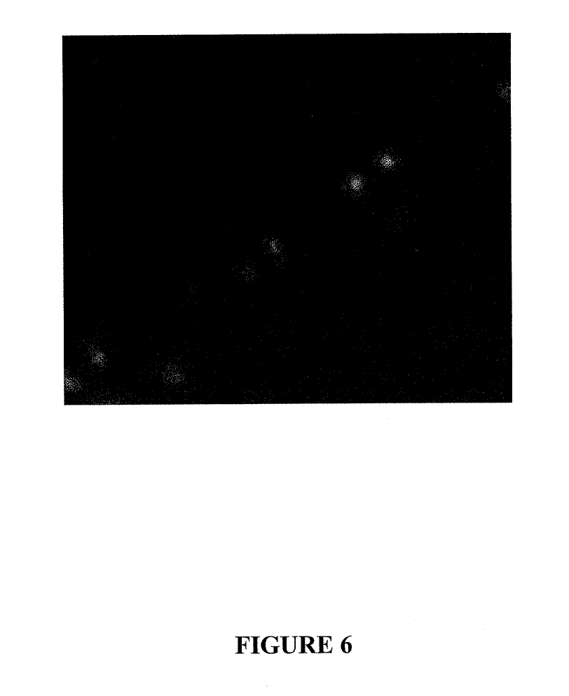

FIG. 6 is an additional view of Purkinje neurons in the same tissue section as photographed in FIG. 5, this time using a 40× microscope objective and fluorescent illumination. This figure provides additional evidence suggesting that the chimeraplasts have entered the nuclei of the Purkinje cells.

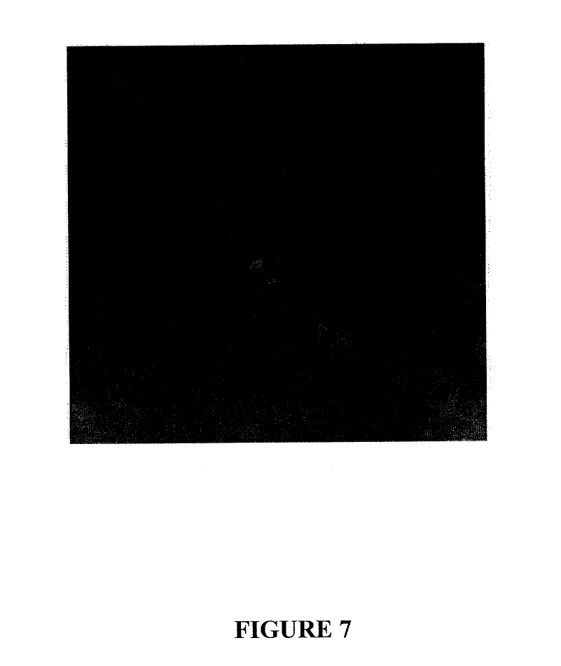

FIGS. 7, 8, and 9 are three views of the same sagittal section of mouse cerebellum, from the same mouse as portrayed in FIGS. 1 through 6. FIG. 7 shows the tissue section under fluorescent illumination, using a 20× microscope objective. The significance of FIG. 7 is that it shows the position of the signal from the fluorescein-labeled chimeraplasts.

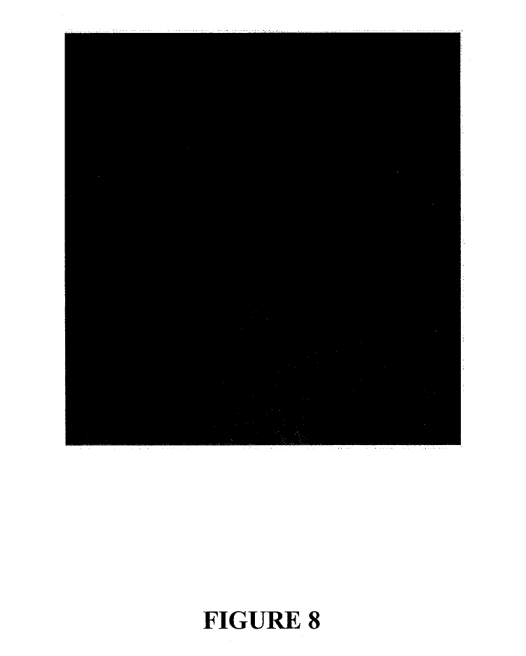

FIG. 8 is the same tissue section as FIG. 7, under fluorescent illumination for the Cy-3 fluorophore. This tissue section has been immunostained for calbindin, a marker for Purkinje neurons, using a primary antibody against calbindin and a Cy-3 conjugated secondary antibody. The significance of this photograph is that it identifies the Purkinje neurons in the tissue by virtue of the Cy-3 signal.

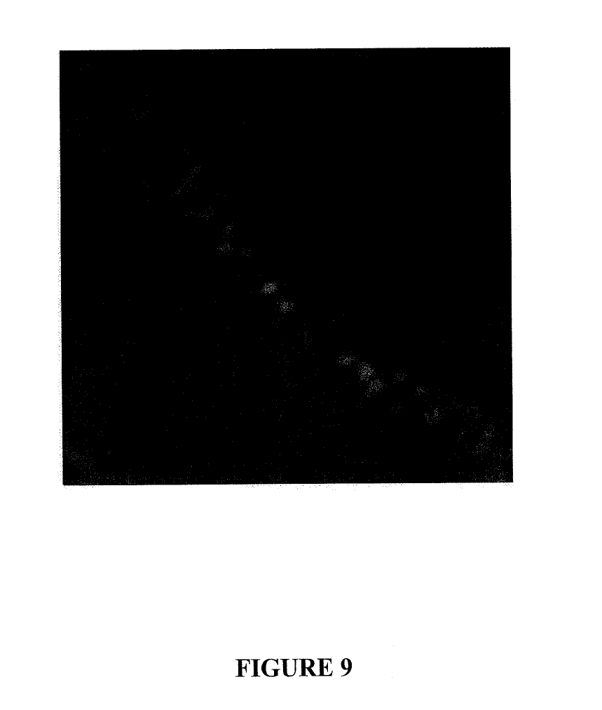

FIG. 9 is the superimposition of FIGS. 7 and 8, indicating that the position of the signals from the fluorescein-labelled chimeraplasts and the signals from the Cy-3/calbindin antibodies are located in the same place. The significance of this figure is that it provides evidence that the neurons that were entered by the chimeraplasts are Purkinje neurons.

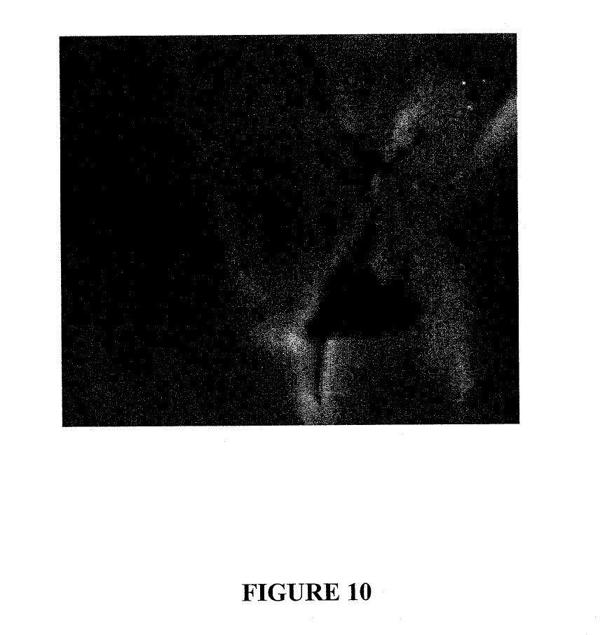

FIG. 10 is a sagittal section of cerebellar tissue from a mouse that had been injected 20 hours earlier with 2 microliters of chimeraplasts at a concentration of 0.6 micrograms per microliter. The significance of this figure is the position of the injection, which can be seen to have been in the molecular (outer) layer of the cerebellar tissue, and the absence of a punctate signal obtained from neurons, indicating that few neurons took up the chimeraplasts when the injection site was in the outer layer of the tissue.

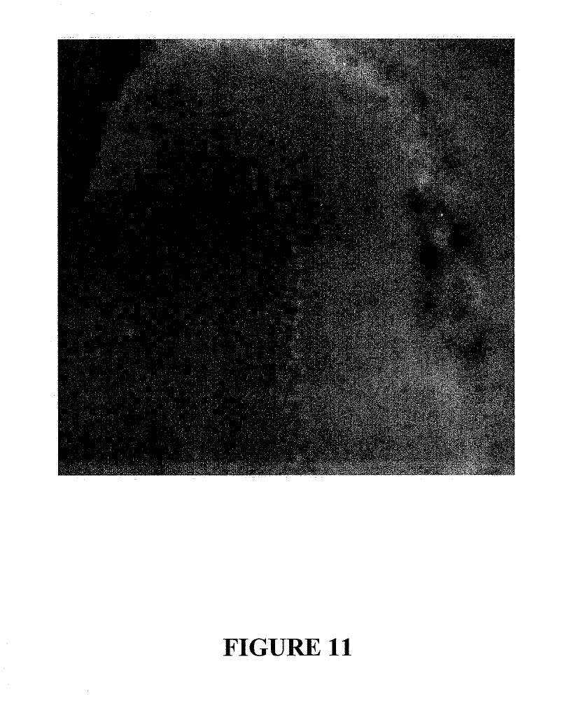

FIG. 11 is a sagittal section of cerebellar tissue from a mouse that had been injected 20 hours earlier with 2 microliters of chimeraplasts at a concentration of 0.06 micrograms per microliter (which is 10 times less than the mouse portrayed in FIG. 10). This photograph, taken using fluorescent illumination and a 10× microscope objective, shows that even at this low concentration, uptake of chimeraplasts into Purkinje neurons is evident. Together with FIG. 10, this figure suggests that the site of injection of the chimeraplasts within the brain tissue can alter the likelihood that the chimeraplasts will enter specific neuronal cell populations.

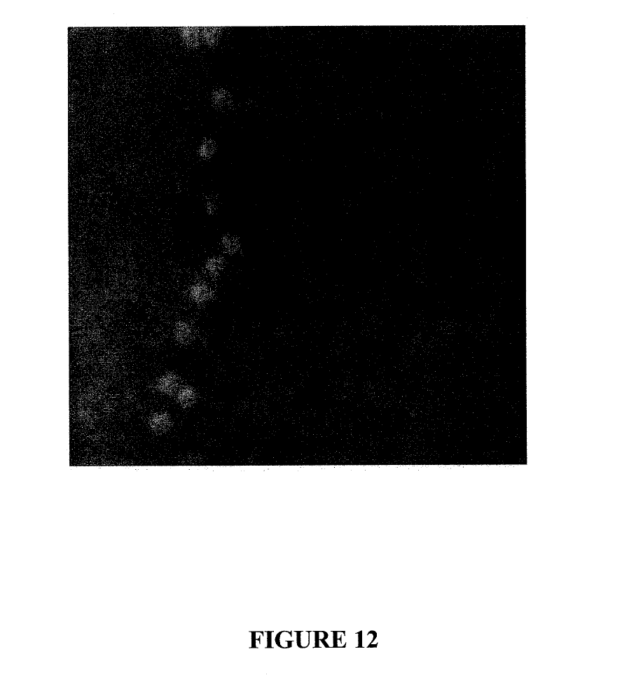

FIG. 12 is a sagittal section of cerebellar tissue from a mouse that had been injected 20 hours earlier with 2 microliters of chimeraplasts at a concentration of 0.06 micrograms per microliter (same as portrayed in FIG. 11). This figure indicates that even at this low concentration, chimeraplasts were taken up by substantial numbers of Purkinje neurons.

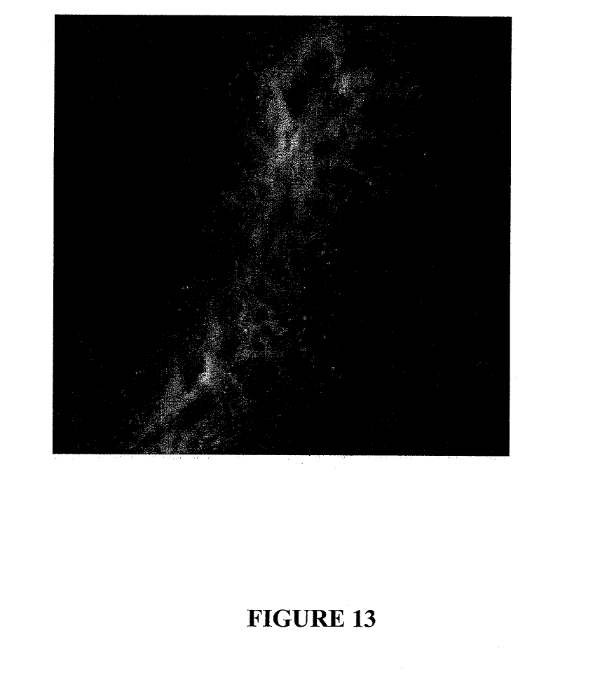

FIG. 13 shows a section of brain tissue from a mouse that had been injected 20 hours earlier with 2 microliters of chimeraplasts at a concentration of 6.0 micrograms per microliter into the striatum. This photograph, taken using fluorescent illumination, indicates that chimeraplasts can be taken up by neurons in the striatum when the striatum is the site of the injection of the chimeraplasts.

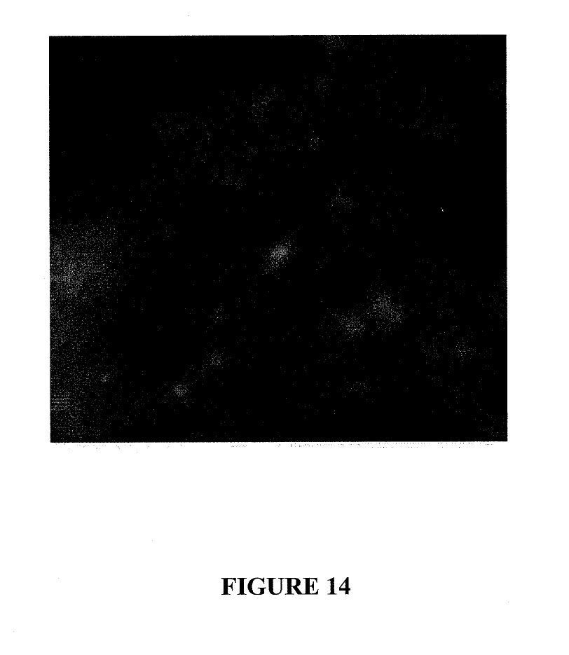

FIG. 14 shows a section of brain tissue from the same mouse as portrayed in FIG. 13, using fluorescent illumination, but using a higher power microscope objective. This figure indicates that the chimeraplasts injected into the striatum are taken up by neurons.

FIG. 15 is a schematic illustration of an example of a catheter for use in a preferred embodiment of the present invention. More specifically, catheter 10 has an access port 12, a strain-relief sleeve 14, and an anchor 16.

FIG. 16 is a schematic illustration of the catheter shown in FIG. 15 when surgically implanted in a patient. More specifically, catheter 10 is shown surgically implanted in patient 18.

Chimeraplasts are a molecular technology that appears capable of engineering single nucleotide changes into the genes of cells. A chimeraplast is an oligonucleotide, approximately 70 to 80 bases long, synthesized to contain both RNA and DNA. The inclusion of RNA in the molecule appears to increase the efficiency with which the oligonucleotide hybridizes with the complementary genomic DNA sequence within a cell (Havre, P. and Kmiec, E. (1998) RecA-mediated joint molecule formation between O-methylated RNA/DNA hairpins and single-stranded targets. Mol Gen Genet 258 (6): 580-586; Gamper, H. J., Cole-Strauss, A., Metz, R., Parekh, H., Kumar, R. and Kmiec, E. (2000) A plausible mechanism for gene correction by chimeric oligonucleotides. Biochemistry 39 (19): 5808-5816).

To target a particular gene, a chimeraplast containing the reverse complement of a portion of the gene's sequence is made. To induce a change in the targeted gene, a single base in the chimeraplast is deliberately designed not to be the correct complement; rather, it is the complement for the nucleotide that is desired. Evidence suggests that when a chimeraplast enters a cell and hybridizes with its target gene, the resulting mismatch becomes a substrate for DNA mis-match repair enzymes (Cole-Strauss, A., Gamper, H., Holloman, W., Munoz, M., Cheng, N. and Kmiec, E. (1999) Targeted gene repair directed by the chimeric RNA/DNA oligonucleotide in a mammalian cell-free extract. Nucleic Acids Research 27 (5): 1323-1330). Half of the time, the repair will “correct” the gene sequence to match the chimeraplast, rather than correct the chimeraplast to match the gene.

It has been shown that chimeraplast molecules produce the predicted changes in gene sequences in cells both in vitro (Kren, B., Cole-Strauss, A., Kmiec, E. and Steer, C. (1997) Targeted nucleotide exchange in the alkaline phosphatase gene of HuH-7 cells mediated by a chimeric RNA/DNA oligonucleotide. Hepatology 25 : 1462-1468) and in vivo (Kren, B., Bandyopadhyay, P. and Steer, C. (1998) In vivo site-directed mutagenesis of the factor IX gene by chimeric RNA/DNA oligonucleotides. Nature Medicine 4: 285-290). Furthermore, the effects are long lasting (Kren, B., Bandyopadhyay, P., Chowdhury, N., Chowdhury, J. and Steer, C. (1999) Correction of the UDP-glucuronosyltransferase gene defect in the Gunn rat model of Crigler-Najjar syndrome type 1 with a chimeric oligonucleotide. Proceedings of the National Academy of Sciences USA 96: 10349-10354) and can be therapeutic. Chimeraplasts can correct an inherited single-point mutation in the gene for an essential liver enzyme in a rat model of Crigler-Najjar syndrome. In this disease, the inherited deficiency in the liver enzyme results in a build-up of excess bilirubin. In the rat model, multiple intravenous administrations of a chimeraplast designed to repair the mutation resulted in changes in liver DNA and reduction in serum bilirubin levels. These changes persisted for at least six months after the treatment (Kren, B., Bandyopadhyay, P., Chowdhury, N., Chowdhury, J. and Steer, C. (1999) Correction of the UDP-glucuronosyltransferase gene defect in the Gunn rat model of Crigler-Najjar syndrome type 1 with a chimeric oligonucleotide. Proceedings of the National Academy of Sciences USA 96: 10349-10354).

There are various ways that a chimeraplast strategy could be used to suppress ataxin-1 protein production. Ataxin-1 is the protein that when mutated causes spinocerebellar ataxia type 1. A chimeraplast might be used for site-directed mutagenesis of the nuclear localization signal in ataxin-1. See Klement, I., Skinner, P., Kaytor, M., Yi, H., Hersch, S., Clark, H., Zoghbi, H. and Orr, H. (1998) Ataxin-1 nuclear localization and aggregation: role in polyglutamine-induced disease in SCA1 transgenic mice. Cell 95 (1): 41-53 for the finding that mutation of this signal prevents ataxin-1 from translocating to the cell nucleus and averts pathogenesis in Purkinje cells. To the extent that some normal functions of ataxin-1 occur in the cytoplasm, the strategy would preserve some normal function while preventing pathology. Alternatively, insertion or deletion of a nucleotide into ataxin-1 sequence could produce a frame-shift resulting in a nonsense mutation. More “cleanly,” ataxin-1 production might be suppressed by changing a nucleotide to produce a premature stop codon. The stop codon or frame-shift should be introduced prior to the CAG repeat region, since evidence from various models and cell culture studies indicates that polyglutamine-containing protein fragments are themselves cytotoxic (see Ellerby, L., Andrusiak, R., Wellington, C., Hackam, A., Propp, S., Wood, J., Sharp, A., Margolis, R., Ross, C., Salvesen, G., Hayden, M. and Bredesen, D. (1999) Cleavage of atrophin-1 at caspase site aspartic acid 109 modulates cytotoxicity. J Biol Chem 274 (13): 8730-8736; and Faber, P., Alter, J., MacDonald, M. and Hart, A. (1999) Polyglutamine-mediated dysfunction and apoptotic death of a caenorhabditis elegans sensory neuron. Proc Natl Acad Sci USA 96 (1): 179-184).

A prerequisite for any of these approaches to therapy for SCA1 will be the ability to deliver chimeraplasts into Purkinje cells and other neurons in vivo. The following study was performed as an initial test of this ability.

Materials and Methods:

Fluorescein-conjugated chimeric oligonucleotides were kindly provided by the University of Minnesota. Because the goal of this study was only a short-term assessment of whether chimera enter Purkinje cells when delivered in vivo, the specific function of these chimera (designed to alter a β-globulin gene sequence) was irrelevant.

Five 4-week old female FVB/N littermates received stereotactic injections of these chimera into the cerebellar cortex at coordinates AP −2.75, ML −1.25, and DV 0.5 mm from lambda, using anesthesia and surgical techniques as described below. The Hamilton syringe tip was dipped in charcoal prior to insertion to allow identification of the injection site in later histology. Two mice received 12 μg, two received 1.2 μg, and one received 0.12 μg in 2 μl volume of sterile culture grade water. Twenty-two hours later, the mice were sacrificed for cerebellar histology as described. The cerebella were cut into 30 μm thick serial sections in the sagittal plane, and every other section was mounted on a glass slide and coverslipped using a 2% solution of gelatin in culture grade water. After sections were examined for chimera entry into cells, selected adjacent sections were immunostained for calbindin and mounted to identify Purkinje cells.

Chimeraplast Injections

Wildtype FVB/N mice were injected intraperitoneally with 6 μl of ketamine/xylazine mixture (36 mg/ml ketamine, 5.5 mg/ml xylazine) to produce deep anesthesia. The mouse was mounted in a stereotactic frame (Kopf Model 900), and its head shaved. A midline sagittal incision was made and the cranium over the right cerebellar hemisphere was exposed. At the injection site, a burr hole was drilled and a Hamilton syringe inserted to the stereotactic coordinates described. The syringe was then advanced an additional 0.25 mm below dura, left in place for 2 minutes, then retracted 0.25 mm, to form a slight pocket in the parenchyma. After a pause of at least 2 minutes for pressure equalization, the injection was performed manually at an approximate rate of 0.5 μl per minute. The total volume injected was 2 μl. After the injection was complete, the syringe was left in place for 3 more minutes, and then withdrawn over a period of 2 minutes or more. The scalp was sutured and the mouse kept under a warming lamp until recovered from the anesthesia then returned to standard housing.

Brain Tissue Processing

Twenty-two hours after the injections, mice were deeply anesthetized by intraperitoneal injection of 12 μl sodium pentobarbital and transcardially perfused with phosphate-buffered saline (PBS) for several minutes, followed by perfusion with 4% formaldehyde for 10 to 15 minutes. The brain was removed and post-fixed for 1 to 2 hours in 4% formaldehyde, then transferred to a 30% solution of sucrose and stored at 4° C. until it sank. Then, the brain was frozen in dry ice, and cut into 30 μm serial sagittal sections using a sliding microtome.

Chimeraplast Detection

For visualization of the fluorescein-conjugated chimeraplasts, tissue sections were rinsed 3×20 minutes in PBS, mounted on glass slides, and coverslipped with a 2% solution of gelatin. They were protected from light while the mounting solution set, then viewed by fluorescence 5 microscopy using filters appropriate for the excitation and emission wavelengths of fluorescein.

Immunohistochemistry

Selected tissue sections from cerebella injected with chimeraplasts were immunostained for calbindin using an anti-calbindin primary antibody and Cy-3 conjugated secondary antibody, as described below.

Sections were rinsed 3×20 minutes in PBS, then transferred to a solution containing 2% normal goat serum (NGS) and 0.3% Triton-X-100 for a minimum of 1 hour. Sections were then transferred to a solution of 2% NGS, 0.3% Triton-X-100 and 1:500 antibody to calbindin-D-28k (Sigma, #C8666), and incubated at 4° C. with gentle agitation for at least 48 hours. Sections were rinsed 3×20 minutes at room temperature in PBS, then incubated for at least 24 hours at 4° C., with gentle agitation, in a solution of 2% NGS, 0.3% Triton-X-100 and 1:400 goat-antimouse IgG antibody conjugated to Cy3 fluorophore (Jackson ImmunoLabs #115-165-146) or 1:400 goat-antimouse IgG antibody conjugated to Cy2 fluorophore (Jackson ImmunoLabs #115-225-146). After incubation with the secondary antibody, sections were washed 3×20 minutes, mounted on slides and coverslipped as described.

Results

Cellular uptake of chimera was detected in both mice that received the highest concentration of chimera (6 μg/μl) and in the mouse that received the lowest concentration (0.06 μg/μl). FIGS. 1-6 show various views of four tissue sections (spanning 630 μM medial-laterally) from a mouse that received the highest concentration of chimera. Intense fluorescence is visible in the region immediately surrounding the injection site (identified by the charcoal residue) and punctate signal is visible in the region of the Purkinje cell layer. In this animal, the syringe tip was positioned in the cerebellar molecular layer at the base of a sulcus, and a substantial amount of the injected solution apparently leaked out the sulcus to the subdural space. Nevertheless, considerable uptake of chimera into cells co-located with the Purkinje cell layer occurred. Higher magnification views reveal a central concentrated area of fluorescein signal within many of these cells, suggesting that the chimera entered the cell nucleus.

FIGS. 7-9 show that the cells in the Purkinje cell layer that took up the chimera in this animal were calbindin immunoreactive, suggesting that the chimera in fact entered Purkinje cells.

Oddly, neither of the two mice injected with the intermediate concentration of chimera showed punctate concentration of the fluorescein signal suggestive of cellular uptake. In particular, FIG. 10 shows the lack of punctate signal despite the apparent injection of the chimera solution directly into the Purkinje cell layer. However, the mouse injected with the lowest concentration (0.06 μg/μl) showed similar, though less intense, punctate fluorescein signal from the Purkinje cell layer as the mice injected with the highest concentration (see FIGS. 11 and 12). In this mouse, as in the other two, the needle tip was positioned in the molecular layer of the cerebellum. Comparison of the signal from this mouse to a region of its Purkinje cell layer that is distal from the injection site confirms that this signal, though weak, was well-above background fluorescence. The signal also co-localized with calbindin immunoreactivity.

These data suggest that in vivo delivery of chimeric oligonucleotides to Purkinje cells is possible, for example by direct injection of the “naked” (i.e., unmodified and unencapsulated) oligos into the cerebellum. The apparent “specificity” of the chimera for Purkinje cells was unexpected, and may be related to the particular site of the injection, which in the mice in which Purkinje cell uptake occurred was in the molecular layer. Because the molecular layer is densely populated by Purkinje cell dendritic arbors, injections to this layer may lead to greater exposure of Purkinje cell surface area to chimera than injections at the Purkinje cell layer itself. Similarly, the total surface area of the highly branched Purkinje cell dendrites is probably orders of magnitude greater than the area of the parallel fibers (granule cell axons). This may account for the apparent lack of granule cell uptake of the chimera. Thus, pending replication of this work with greater numbers of animals, it is hypothesized oligonucleotide injection to the molecular layer of the cerebellum favors Purkinje cell uptake.

A more definitive way to target chimera to a specific cell type is to conjugate chimeric oligonucleotides to a peptide moiety that is a ligand for a receptor on the cell surface. This has been shown to be a viable method for preferentially delivering chimera to hepatocytes in vivo, targeting the asialoglycoprotein receptor (see Bandyopadhyay, P., Ma, X., Linehan-Stieers, C., Kren, B. and Steer, C. (1999) Nucleotide exchange in genomic DNA of rat hepatocytes using RNA/DNA oligonucleotides. Targeted delivery of liposomes and polyethylenimine to the asialoglycoprotein receptor. J. Biol. Chem. 274: 10163-10172). Conjugation of a chimeric oligonucleotide with a peptide that binds to bFGF receptor type 1 may be a way to target chimeraplasts to Purkinje cells.

In accordance with the present invention, it is contemplated that chimeric oligonucleotides can trigger changes in genomic DNA within Purkinje cells or other neurons as they do in hepatocytes (see Kren, B., Bandyopadhyay, P., Chowdhury, N., Chowdhury, J. and Steer, C. (1999a) Correction of the UDP-glucuronosyltransferase gene defect in the Gunn rat model of Crigler-Najjar syndrome type 1 with a chimeric oligonucleotide. Proceedings of the National Academy of Sciences USA 96 : 10349-10354; and Bandyopadhyay, P., Ma, X., Linehan-Stieers, C., Kren, B. and Steer, C. (1999) Nucleotide exchange in genomic DNA of rat hepatocytes using RNA/DNA oligonucleotides. Targeted delivery of liposomes and polyethylenimine to the asialoglycoprotein receptor. J. Biol. Chem. 274: 10163-10172). This can be confirmed with in vitro testing of a chimeric oligo designed to introduce a base change in the normal SCA1 gene sequence, using cell lines that have been stably transfected with SCA1 and human neuroblastoma and medulloblastoma cell lines. A chimeric oligo designed to change the T at position 1147 to a G will, if successful, simultaneously introduce a premature TGA stop codon and a new restriction site for HincII at this position, such that PCR and restriction analysis of DNA isolated from these cells will provide a preliminary test of chimeric activity in neuronal cell lines.

Examples of mutational vectors designed to produce a therapeutic change in the genomic DNA sequence for the human SCA1 gene are provided below. Note that the uppercase versus lowercase letters are important in designating whether the corresponding position in the mutational vector is made from DNA or RNA. The “5′-” and “-3′” notations at the start and end of the lines will be recognized by those skilled in the art as designating the orientation of the oligonucleotide molecules. The “GenBank Accession Number” gives the look-up number needed for someone to retrieve the genomic DNA sequence for the human SCA1 gene from the public database maintained (and made available on-line via the Internet) by the National Library of Medicine.

Example A: Mutational vector designed to change the coding strand of the genomic sequence of the DNA for Spinocerebellar Ataxia Type 1 (SCA1) from T to G at position 1147 in the SCAL gene (GenBank Accession Number X79204). Uppercase letters stand for deoxyribonucleotide bases (A=Adenine, T=Thymine, G=Guanine, C=Cytosine) and lowercase letters stand for ribonucleotide bases (A=Adenine, U=Uracil, G=Guanine, C=Cytosine).

5′- AACCTATTCCCTGTTGTCAACCAAGCTCCACCGAGTTTTcucggug
gagcuuggTTGACaacagggaauagguuGGCGCTTTTGCGCC - 3′

Example B: Mutational vector designed to change the non-coding strand of the genomic sequence of the DNA for Spinocerebellar Ataxia Type 1 (SCA1) from A to C at position 1147 in the SCA1 gene (GenBank Accession Number X79204). Uppercase letters stand for deoxyribonucleotide bases (A=Adenine, T=Thyrnine, G=Guanine, C=Cytosine) and lowercase letters stand for ribonucleotide bases (A=Adenine, U=Uracil, G=Guanine, C=Cytosine).

5′- CTCGGTGGAGCTTGGTTGACAACAGGGAATAGGTTTTTTaaccuau
ucccuguuGTCAAccaagcuccaccgagCCGCCTTTTGGCGG - 3′

The substance used in accordance with the present invention can be combined with any suitable dilution agent, including but not limited, to 5% dextrose.

It is to be understood that various modifications, changes and variations are possible in light of the above teachings without departing from the spirit and scope of this invention, as set forth in the appended claims.

We claim:

1. A method of treating a neurodegenerative disorder comprising the steps of:
surgically implanting an intraparenchymal catheter having a port so that a discharge portion of the catheter lies adjacent a predetermined infusion site in a brain; and
discharging through the discharge portion of the catheter a predetermined dosage of at least one substance to the infusion site of the brain, the at least one substance capable of altering a nucleotide in a DNA sequence of a gene to convert a codon in a protein-coding region of the gene into a stop codon in the brain, whereby neurodegeneration in the brain is reduced.

2. The method of claim 1, wherein said step of implanting the catheter is performed after the neurodegenerative disorder is diagnosed.

3. The method of claim 1 further comprising the steps of:
implanting the pump outside the brain, the pump coupled to a proximal end of the catheter; and
operating the pump to deliver the predetermined dosage of the at least one substance from through the discharge portion of the catheter.

4. The method of claim 3 further comprising the step of periodically refreshing the pump with the at least one substance.

5. The method of claim 1, wherein the at least one substance is a mutational vector.

6. The method of claim 5, wherein the at least one substance is a RNA/DNA chimeric mutational vector.

7. The method of claim 1, wherein the neurodegenerative disorder comprises Huntington's disease, spinocerebellar ataxia type 1, type 2, type 3, type 6, and/or type 7, spinobulbar muscular atrophy (Kennedy's disease), and/or dentatorubral-pallidoluysian atrophy (DRPLA).

8. The method of claim 3, wherein the at least one substance is a mutational vector.

9. The method of claim 8, wherein the at least one substance is a RNA/DNA chimeric mutational vector.

10. The method of claim 3, wherein the neurodegenerative disorder comprises Huntington's disease, spinocerebellar ataxia type 1, type 2, type 3, type 6, and/or type 7, spinobulbar muscular atrophy (Kennedy's disease), and/or dentatorubral-pallidoluysian atrophy (DRPLA).

11. The method of claim 4, wherein the at least one substance is a mutational vector.

12. The method of claim 11, wherein the at least one substance is a RNA/DNA chimeric mutational vector.

13. The method of claim 4, wherein the neurodegenerative disorder comprises Huntington's disease, spinocerebellar ataxia type 1, type 2, type 3, type 6, and/or type 7, spinobulbar muscular atrophy (Kennedy's disease), and/or dentatorubral-pallidoluysian atrophy (DRPLA).
